(19) United States
(12) Patent Application Publication
(10) Pub. No.: US 2020/0323159 A1
(43) Pub. Date: Oct. 15, 2020

(54) METHODS TO INCREASE MAIZE HYBRID SEED PRODUCTION EFFICIENCY

(71) Applicants: E. I. DU PONT DE NEMOURS AND COMPANY, WILMINGTON, DE (US); PIONEER HI-BRED INTERNATIONAL, INC., JOHNSTON, IA (US)

(72) Inventors: DARIA SCHMIDT, POLK CITY, IA (US); AARON WALTZ, JANESVILLE, WI (US)

(73) Assignees: E. I. DU PONT DE NEMOURS AND COMPANY, WILMINGTON, DE (US); PIONEER HI-BRED INTERNATIONAL, INC., JOHNSTON, IA (US)

(21) Appl. No.: 16/913,254

(22) Filed: Jun. 26, 2020

Related U.S. Application Data
(63) Continuation of application No. 14/902,702, filed on Jan. 4, 2016.
(60) Provisional application No. 61/982,924, filed on Apr. 23, 2014.
(60) Provisional application No. 61/841,996, filed on Jul. 2, 2013.

(51) Int. Cl. A01H 1/02; A01H 5/10; A01H 1/04; A01C 14/00

(52) U.S. Cl. CPC A01H 1/02; A01H 5/10; Y02A 40/14; A01C 14/00; A01H 1/04

(57) ABSTRACT

The discovery is in the field of plant breeding and improvement, related specifically to a hybrid field corn seed production method that utilizes floret development variant donors as female inbreds or modified inbreds. The methods increase yield in a production field and speeds and aids production. Also provided are field corn ears with the floret development phenotype and seed from the ears.

### CROSS REFERENCE TO RELATED APPLICATIONS

This application claims the benefit of U.S. Provisional Application No. 61/841,996, filed Jul. 2, 2013, and U.S. Provisional Application No. 61/982,924, filed Apr. 23, 2014, the entire contents of which are hereby incorporated by reference.

### FIELD

The field is in the area of plant breeding as it relates to hybrid seed production for field corn.

### BACKGROUND

Commercial seed production is a complex process that involves many steps. In the case of corn, for example, inbred plants must first be crossed in a field under conditions that allow the male pollen to fertilize the silks on female ears in order to produce seed that will be sold to the farmer. The quality of the seed produced is critical in the marketplace, and many factors affect seed quality. For example, variations in size, moisture content, and ripeness at the upstream end of the process influence the effectiveness of each step in the seed production process and can produce substantial variability in the end product and/or may trigger a need for adjustments in operating parameters further downstream.

Seed production is a limiting factor in many crop species, in particular in hybrid crop species. In addition, over one thousand square miles are used for seed production each year. Increases in production efficiency can reduce the amount of production field required, thus freeing fields to be used in food and feed production, and can lead to significant cost reductions.

Accordingly, there is a need in the art for an improved system and method of seed production that allows for more consistently sized seed to be produced in order to optimize the quality of seed produced in an efficient and cost-effective manner.

### SUMMARY

Compositions and methods involving field corn hybrid seed production are presented herein. Field corn female (inbred or modified inbred in the case of three-way or double cross hybrids) ears that exhibit non-uniform rowing and uniform seeds of small to medium size and flat shape (in line with a floret development variant phenotype) are provided, as are seed of the ears. The seed may comprise a seed treatment.

Methods of hybrid seed production in field corn are presented herein. The methods comprise planting floret development variants as females in a hybrid corn production field, harvesting ears from the floret development variant donors; and obtaining hybrid field corn seed from the ears. The floret development variant donors may be inbreds. Furthermore, the floret development variant donors may be detasseled prior to anthesis or may be male-sterile.

The methods described herein have other advantages in the seed production process and hence, alternative methods may include: methods for producing uniform seed lots of small to medium flat seeds, methods for increasing seed production yield, methods for minimizing post-harvest processing damage to the seed, methods for reducing seed drying time, methods for facilitating the removal of seed from the ears, methods for decreasing seed processing equipment wear and tear and maintenance, methods for reducing or eliminating the need for seed size sorting, methods for increasing seed treatment uniformity and efficiency, methods for increasing germ plasm security, methods for increasing transportation efficiency of commercial seed, methods of increasing planting efficiency, methods of decreasing seed processing plant biological refuse, methods of increasing efficiency of color sorting of seed, and/or methods for increasing seed germination and stand count.

Also provided are methods of increasing the efficiency of hybrid corn production by crossing a male and a female plant in which the female corn plant comprises one or more QTL alleles associated with the floral development variant phenotype at any of the QTL positions listed in Table 7, 8, 9, 10 or 11 and wherein the male corn plant does not have the QTL allele associated with the floret development variant phenotype.

Also provided is a method of creating the production female by introgressing a QTL allele associated with the floret development variant phenotype into a corn plant that does not comprise the QTL allele in its genome, said method comprising: A) providing at least a corn plant, B) genotyping at least one corn plant at one or more marker loci in any of the QTL regions listed in Table 7, 8, 9, 10, or 11, and, C) selecting at least one corn plant for further analysis or breeding based upon the presence of an allele at the at least one marker locus that is correlated with the QTL allele associated with the floret development variant phenotype.

### BRIEF DESCRIPTION OF THE DRAWINGS

The invention can be more fully understood from the following detailed description and the accompanying drawings which form a part of this application.

FIG. 1 shows 1000 gram seed distribution across standard sizing screens. The Y-axis is in grams. The X-axis lists the standard screen sizes from large seed (26) to “less than 14”.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, and reference to “a cell” includes one or more cells and equivalents thereof known to those skilled in the art, and so forth.

As used herein:

A “plant” can be a whole plant, any part thereof, or a cell or tissue culture derived from a plant. Thus, the term “plant” can refer to any of: whole plants, plant components or organs (e.g., leaves, stems, roots, etc.), plant tissues, seeds, plant cells, and/or progeny of the same. A plant cell is a cell of a plant, taken from a plant, or derived through culture from a cell taken from a plant.

The term “maize plant” includes whole maize plants, maize plant cells, maize plant protoplast, maize plant cell or maize tissue culture from which maize plants can be regenerated, maize plant calli, maize plant clumps and maize plant cells that are intact in maize plants or parts of maize plants, such as maize seeds, maize cobs, maize flowers, maize cotyledons, maize leaves, maize stems, maize buds, maize roots, maize root tips and the like.

The term “maize” includes any member of the species Zea mays. “Maize” and “corn” are used interchangeably herein.

A “seed” is a small embryonic plant enclosed in a protective seed coat. It is the product of the ripened plant ovule generated after fertilization.

“Germplasm” refers to genetic material of or from an individual (e.g., a plant), a group of individuals (e.g., a plant line, variety, or family), or a clone derived from a line, variety, species, or culture. The germplasm can be part of an organism or cell, or can be separate from the organism or cell. In general, germplasm provides genetic material with a specific molecular makeup that provides a physical foundation for some or all of the hereditary qualities of an organism or cell culture. As used herein, germplasm includes cells, seed or tissues from which new plants may be grown, or plant parts, such as leafs, stems, pollen, or cells, that can be cultured into a whole plant.

“Variety”, when used in conjunction with plants, encompasses botanical and cultivated plants, including inbreds and hybrids, and means a plant grouping within a single botanical taxon of the lowest known rank, where the grouping can be defined by the expression of characteristics resulting from a given genotype or combination of genotypes.

The term “inbred” means a substantially homozygous variety.

The term “modified inbred” means a variety used to produce hybrid commercial seed.

The term “hybrid” means any offspring/progeny of a cross between two genetically unlike individuals, including a cross of two different inbred lines, one modified inbred and one inbred line or two modified inbred lines.

The term “allele” refers to one of two or more different nucleotide sequences that occur at a specific locus.

An allele is “associated with” a trait when it is linked to it and when the presence of the allele is an indicator that the desired trait or trait form will occur in a plant comprising the allele.

A “favorable allele” is the allele at a particular locus that confers, or contributes to, an agronomically desirable phenotype, e.g., floret development, or alternatively, is an allele that allows the identification of susceptible plants that can be removed from a breeding program or planting. A favorable allele of a marker is a marker allele that segregates with the favorable phenotype, or alternatively, segregates with susceptible plant phenotype, therefore providing the benefit of identifying disease-prone plants. A favorable allelic form of a chromosome segment is a chromosome segment that includes a nucleotide sequence that contributes to superior agronomic performance at one or more genetic loci physically located on the chromosome segment.

“Allele frequency” refers to the frequency (proportion or percentage) at which an allele is present at a locus within an individual, within a line, or within a population of lines. For example, for an allele “A”, diploid individuals of genotype “AA”, “Aa”, or “aa” have allele frequencies of 1.0, 0.5, or 0.0, respectively. One can estimate the allele frequency within a line by averaging the allele frequencies of a sample of individuals from that line. Similarly, one can calculate the allele frequency within a population of lines by averaging the allele frequencies of lines that make up the population. For a population with a finite number of individuals or lines, an allele frequency can be expressed as a count of individuals or lines (or any other specified grouping) containing the allele.

An allele “positively” correlates with a trait when it is linked to it and when presence of the allele is an indicator that the desired trait or trait form will occur in a plant comprising the allele. An allele “negatively” correlates with a trait when it is linked to it and when presence of the allele is an indicator that a desired trait or trait form will not occur in a plant comprising the allele.

An individual is “homozygous” if the individual has only one type of allele at a given locus (e.g., a diploid individual has a copy of the same allele at a locus for each of two homologous chromosomes).

An individual is “heterozygous” if more than one allele type is present at a given locus (e.g., a diploid individual with one copy each of two different alleles).

A special case of a heterozygous situation is where one chromosome has an allele of a gene and the other chromosome lacks the gene, locus, or region completely—in other words, has a deletion relative to the first chromosome. This situation is referred to as “hemizygous”.

The term “homogeneity” indicates that members of a group have the same genotype at one or more specific loci. In contrast, the term “heterogeneity” is used to indicate that individuals within the group differ in genotype at one or more specific loci.

A “locus” is a chromosomal region where a polymorphic nucleic acid, trait determinant, gene, or marker is located. Thus, for example, a “gene locus” is a specific chromosome location in the genome of a species where a specific gene can be found. A locus correlated with floret development denotes a region on the genome that is directly related to a phenotypically quantifiable floret development trait.

A “genetic complement” has at least one set or ploidy of alleles. For example, a single cross hybrid inherits two genetic complements, one from each inbred parent.

The term “quantitative trait locus” or “QTL” refers to a polymorphic genetic locus with at least one allele that correlates with the differential expression of a phenotypic trait in at least one genetic background, e.g., in at least one breeding population or progeny. A QTL can act through a single gene mechanism or by a polygenic mechanism.

A “polymorphism” is a variation in the DNA that is too common to be due merely to new mutation (i.e. occurs at a frequency of at least 1% in a population). Any differentially inherited polymorphic trait (including nucleic acid polymorphism) that segregates among progeny is a potential marker. The genomic variability can be of any origin, for example, insertions, deletions, duplications, repetitive elements, point mutations, recombination events, or the presence and sequence of transposable elements.

As used herein, the term “crossed” or “cross” refers to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

The term “introgression” refers to the transmission of a desired allele of a genetic locus from one genetic background to another. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., a selected allele of a marker, a QTL, a transgene, or the like. In any case, offspring comprising the desired allele can be repeatedly backcrossed to a line having a desired genetic background and selected for the desired allele, to result in the allele becoming fixed in a selected genetic background.

The process of “introgressing” is often referred to as “backcrossing” when the process is repeated two or more times.

A “backcross conversion” is a product of introgression of a locus or trait into a variety by backcrossing.

“Backcrossing” refers to the process whereby hybrid progeny are repeatedly crossed back to one of the parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. (1995) Marker-assisted backcrossing: a practical example, in Techniques et Utilisations des Marqueurs Moleculaires Les Colloques, Vol. 72, pp. 45-56, and Openshaw et al., (1994) Marker-assisted Selection in Backcross Breeding, Analysis of Molecular Marker Data, pp. 41-43. The initial cross gives rise to the F₁ generation; the term “BC1” then refers to the second use of the recurrent parent, “BC2” refers to the third use of the recurrent parent, and so on.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor.

An “ancestral line” is a parent line used as a source of genes e.g., for the development of elite lines. An “ancestral population” is a group of ancestors that have contributed the bulk of the genetic variation that was used to develop elite lines. “Progeny” are the descendants of ancestors, and may be separated from their ancestors by many generations of breeding. For example, elite lines are the progeny of their ancestors. A “pedigree structure” defines the relationship between a progeny and each ancestor that gave rise to that descendant. A pedigree structure can span one or more generations, describing relationships between the progeny and its parents, grandparents, great-grand parents, etc.

An “elite line” or “elite strain” is an agronomically superior line that has resulted from many cycles of breeding and selection for superior agronomic performance. Numerous elite lines are available and known to those of skill in the art of maize breeding. An “elite population” is an assortment of elite individuals or lines that can be used to represent the state of the art in terms of agronomically superior genotypes of a given crop species, such as maize. Similarly, an “elite germ plasm” or elite strain of germplasm is an agronomically superior germplasm, typically derived from and/or capable of giving rise to a plant with superior agronomic performance, such as an existing or newly developed elite line of maize.

In contrast, an “exotic maize strain” or an “exotic maize germplasm” is a strain or germplasm derived from maize that does not belong to an available elite maize line or strain of germ plasm. In the context of a cross between two maize plants or strains of germplasm, an exotic germplasm is not closely related by descent to the elite germplasm with which it is crossed. Most commonly, the exotic germplasm is not derived from any known elite line of maize, but rather is selected to introduce novel genetic elements (typically novel alleles) into a breeding program.

The term “genetic element” or “gene” refers to a heritable sequence of DNA, i.e., a genomic sequence, with functional significance. The term “gene” can also be used to refer to, e.g., a cDNA and/or an mRNA encoded by a genomic sequence, as well as to that genomic sequence.

The term “genotype” is the genetic constitution of an individual (or group of individuals) at one or more genetic loci, as contrasted with the observable trait (the phenotype). Genotype is defined by the allele(s) of one or more known loci that the individual has inherited from its parents. The term genotype can be used to refer to an individual's genetic constitution at a single locus, at multiple loci, or, more generally, the term genotype can be used to refer to an individual's genetic make-up for all the genes in its genome.

The terms “phenotype”, or “phenotypic trait” or “trait” refers to one or more trait of an organism. The phenotype can be observable to the naked eye, or by any other means of evaluation known in the art, e.g., microscopy, biochemical analysis, genomic analysis, or an assay for a particular disease tolerance. In some cases, a phenotype is directly controlled by a single gene or genetic locus, i.e., a “single gene trait”. In other cases, a phenotype is the result of several genes.

The term “yield” refers to the productivity per unit area of a particular plant product of commercial value. For example, yield of maize is commonly measured in bushels of seed per acre or metric tons of seed per hectare per season. Yield is affected by both genetic and environmental factors. “Agronomics”, “agronomic traits”, and “agronomic performance” refer to the traits (and underlying genetic elements) of a given plant variety that contribute to yield over the course of growing season. Individual agronomic traits include emergence vigor, vegetative vigor, stress tolerance, disease resistance or tolerance, herbicide resistance, branching, flowering, seed set, seed size, seed density, standability, threshability, and the like. Yield is, therefore, the final culmination of all agronomic traits.

“Silks” are the structures that emerge from the ear shoot and are the functional stigmas of the female flowers of a corn plant. Each silk connects to an individual ovule (potential kernel). A given silk must be pollinated in order for the ovule to be fertilized and develop into a kernel.

Plants to be used as “males” in the seed production process supply pollen to the female plants; the ears produced on male plants are not harvested.

Plants to be used as “females” in the seed production process do not supply pollen either due to removal of the tassel prior to pollination, the presence of genes that confer cytoplasmic male sterility, and/or the application of chemicals (i.e. male gametocides) to control pollen viability or production. The seed produced on ears of the female plants is hybrid seed and is harvested in the seed production process.

The embodiments and aspects discussed herein are described in conjunction with systems, tools and methods which are meant to be exemplary and illustrative, and not limiting in scope. In various embodiments, one or more of the problems associated with seed production and/or seed quality have been reduced or eliminated, while other embodiments are directed to process improvements.

Field crops are bred through techniques that take advantage of the plant's method of reproduction and variation related thereto. In the practical application of a breeding program, the breeder often initially selects and crosses two or more parental lines, followed by repeated selfing and selection, thereby producing many unique genetic combinations. The breeder can theoretically generate billions of different genetic combinations via crossing, selfing, and mutagenesis. However, the breeder commonly has no direct control at the cellular level of the plant. Therefore, two breeders will never independently develop the same variety having the same traits.

In each cycle of evaluation, the plant breeder selects the germ plasm to advance to the next generation. This germ plasm is grown in one or more chosen environments, and further selections are made during and at the end of the growing season.

Once varieties are selected, production techniques are used to manage the creation of the hybrid, which will be sold into the marketplace. In the case of corn, male and female plants (e.g. inbreds or modified inbreds) are generally planted in a field with one or more rows of female plants per male row. The female plants are detasseled prior to pollen production (anthesis) thus causing the pollen from the male row to fertilize the females in the female row(s). Alternatively, the females may be male-sterile (either due to genes that confer cytoplasmic male sterility or male sterility may be induced via chemical means). In any case, the goal is to create hybrid corn wherein the male pollen fertilizes the ovules on the female ears, thus producing hybrid seed. The seed is then harvested, dried, stored, conditioned, and packaged. The post-harvest activities may also involve transportation to a processing plant or other location.

Seed production is a complex process, the result of which is seed that can be sold in the marketplace. There is a need for improvements in seed quality as well as improvements in the seed production process itself that not only maximize output of quality seed but also minimize operational input.

The methods provided herein can be used to increase seed quality at or near the time of harvest or even by storage thereafter. The methods make use of floret development variant donors, or plants that exhibit a floret development variant phenotype. The “floret development variant phenotype” is the result of development of both the lower and upper florets on the spikelets of an ear, leading to a loss of rows and up to twice as many kernels per ear as would typically occur. The kernels are uniform, small to medium in size, and flat in shape. The phenotype is maternally inherited. As such, a female used as a floret development variant donor will exhibit the floret development variant phenotype even if the resulting seed is heterozygous at one or more genetic loci controlling the floret development variant phenotype.

As used herein, a “floret development variant donor” refers to a plant having the floret development variant phenotype. One example of a floret development variant donor is Country Gentleman (CG), a well-known open-pollinated sweet corn variety. Country Gentleman exhibits the floret development variant phenotype yet has normal tassel development. Because the desired consumer end-product in sweet corn is a small, dainty kernel, the floret development variant donor is used as male and/or female in the seed production process.

The methods provided herein describe the use of a floret development variant donor in the production of hybrid seed for field corn. The floret development variant donor may be a field corn plant (e.g. an inbred) derived from Country Gentleman that retains the floret development variant phenotype of Country Gentleman (i.e. has one or more favorable alleles at one or more genetic loci that confer the floret development phenotype).

Until now, no one has appreciated the use of this phenotype to improve seed production in field corn, and this novel and unanticipated use can revolutionize the seed industry.

Hence, methods of hybrid seed production in field corn are presented herein. The methods comprise planting floret development variant donors as females in a hybrid corn production field; harvesting the ears of the females; and obtaining the hybrid field corn seed from the ears. The floret development variant donors used as females may be inbreds. Furthermore, the floret development variant donors may be detasseled prior to anthesis or may be male-sterile.

As used herein, “field corn” refers to varieties or cultivars of corn grown extensively on large acreages within a broad but defined geographic area for the production of grain and/or forage. Most field corn in the United States is also referred to as “dent” corn, whereas field corn produced in Europe and Argentina is more likely to be referred to as “flint” or “flint-dent” corn.

Field corn ears with non-uniform rowing and uniform seeds of small to medium size and flat shape are also provided, as are seed of the field corn ears. The seed may further be treated with any of the seed treatments for crop seed that are described herein.

### Seed and Yield Modifications

A lack of seed size uniformity is a hindrance to producers and farmers as it decreases efficiency at all stages of production. Differences in seed size and shape are due to the position of the kernel on the ear. Small rounds tend to come from the tip of the ear; large rounds tend to come from the butt of the ear; and flats tend to come from the middle of the ear.

When floret development variant donors are used as females in hybrid corn production, the kernels that develop are more uniform across the ear. The methods described herein generate seeds that are more uniform in size and shape. As a result of using floret development variant donors as females, the changes in size and shape of the seed produced also increase seed production yield. Thus, methods for producing uniform seed lots of small to medium sized and flat shaped seeds are provided herein. Also provided are methods of increasing seed production yield as compared to normal hybrid seed production that does not utilize floret development variant donors as females.

### Improvements in the Production Process

Harvested seed may be transported to a processing plant and/or other area for any of the following activities: drying, storage, conditioning, and packaging.

Large sized seeds are a limitation in commercial seed production for many crop species. Larger seeds tend to be more prone to damage during processing, seed treatment, and packaging. Hence, the methods provided herein that use floret development variants as females during hybrid seed production are also methods of increasing seed quality and/or methods of decreasing damage to the seed during post-harvest activities.

Seed corn is usually harvested on the ear and at high moisture content; thus, it is necessary to reduce seed moisture content for safe storage. This process is referred to as “drying”. In the process of drying, heated air is forced through bins containing shelled seed or ears with the seeds still attached. Because the seed produced from floret development variant donors used as females is more uniform, small to medium sized, and flat in shape, the seed may already have low moisture, thereby reducing damage from heat injury (i.e. increasing seed quality). Moreover, the size, shape, and uniformity of the seed produced may allow for increased airflow between the seeds, reducing drying time.

Hence, methods for reducing drying time of harvested seed and/or increasing seed quality are provided herein. The methods involve using plants having the floret development variant phenotype as females in the seed production process, harvesting the seed produced on the female ears, and drying the seed to obtain optimal moisture content. The characteristics of the seed produced on the ears of the females reduce the length of time needed for the drying process thereby increasing seed quality due to reduced exposure to heat.

Seed conditioning begins with shelling of the seed, or the removal of the seed from the ears, and can include three possible operations: the separation of cobs, husks, silks and other debris from the seed; the sorting of the seed into uniform lots based on size and shape; and the treatment of seed prior to packaging.

When floret development variants are used as females in hybrid seed production, the seeds are easier to remove from the ears. Hence, methods of facilitating the removal of seed from the ears using floret development variants as females during the hybrid seed production process are also provided herein. The greater ease of seed shelling may also decrease seed processing plant equipment wear and tear and maintenance.

In addition, when floret development variants are used as females in hybrid seed production, the seeds are more uniform, eliminating the need for sorting of the seed based on size and shape during seed conditioning. Hence, methods of reducing the need for seed size sorting during the conditioning process are also provided.

Uniformity of seed size and shape also enhances seed treatment application by allowing more even coverage and reducing the time and resources needed for application. To protect and to enhance yield production and trait technologies, seed treatment options can provide additional crop plan flexibility and cost effective control against insects, weeds and diseases, thereby further enhancing the invention described herein. Seed material can be treated, typically surface treated, with a composition comprising combinations of chemical or biological herbicides, herbicide safeners, insecticides, fungicides, germination inhibitors and enhancers, nutrients, plant growth regulators and activators, bactericides, nematicides, avicides and/or molluscicides. These compounds are typically formulated together with further carriers, surfactants or application-promoting adjuvants customarily employed in the art of formulation. The coatings may be applied by impregnating propagation material with a liquid formulation or by coating with a combined wet or dry formulation. Examples of the various types of compounds that may be used as seed treatments are provided in The Pesticide Manual: A World Compendium, C. D. S. Tomlin Ed., Published by the British Crop Production Council, which is hereby incorporated by reference.

Some seed treatments that may be used on crop seed include, but are not limited to, one or more of abscisic acid, acibenzolar-S-methyl, avermectin, amitrol, azaconazole, azospirillum, azadirachtin, azoxystrobin, bacillus spp. (including one or more of cereus, firmus, megaterium, pumilis, sphaericus, subtilis and/or thuringiensis), bradyrhizobium spp. (including one or more of betae, canariense, elkanii, iriomotense, japonicum, liaonigense, pachyrhizi and/or yuanmingense), captan, carboxin, chitosan, clothianidin, copper, cyazypyr, difenoconazole, etidiazole, fipronil, fludioxonil, fluquinconazole, flurazole, fluxofenim, harpin protein, imazalil, imidacloprid, ipconazole, isoflavenoids, lipo-chitooligosaccharide, mancozeb, manganese, maneb, mefenoxam, metalaxyl, metconazole, PCNB, penflufen, penicillium, penthiopyrad, permethrine, picoxystrobin, prothioconazole, pyraclostrobin, rynaxypyr, S-metolachlor, saponin, sedaxane, TCMTB, tebuconazole, thiabendazole, thiamethoxam, thiocarb, thiram, tolclofos-methyl, triadimenol, trichoderma, trifloxystrobin, triticonazole and/or zinc. PCNB seed coat refers to EPA registration number 00293500419, containing quintozen and terrazole. TCMTB refers to 2-(thiocyanomethylthio) benzothiazole.

Hence, methods of increasing seed treatment uniformity and efficiency using the floret development variant donors as females are also provided herein.

The methods described herein may also be used to increase germplasm security, to increase transportation efficiency of commercial seed, to increase planting efficiency, to decrease seed processing plant biological refuse, to increase efficiency of color sorting of seed, and/or to increase seed germination and stand count.

With respect to increased germ plasm security, one of ordinary skill in the art will be able to determine if others have used their germ plasm because selfing of the hybrid will produce ears with aberrant rows when the methods described herein have been applied.

Changes in seed size and shape (Example 3) resulting from utilization of the methods described herein leads to less damage to the seed itself (as compared to hybrid seed obtained from more traditional seed production practices, particularly in the case of round shaped larger seeds). Hence, planting efficiency and the efficiency of transportation of the seed can both be increased because a greater percentage of seed will be intact, whole seeds. Also due to the changes in seed size and shape, the efficiency of color sorting of seed can be increased because coloration is more visible in smaller kernels or seeds. Thus, seed can be sorted more easily due to the presence or absence of coloration, whether or not that coloration is due to the expression of a color marker or even due to diseased or moldy seed. In the latter, diseased or moldy seed can be removed from the seed lot upon sorting. Finally, the greater percentage of intact, whole seeds will increase seed germination and/or stand count as compared to hybrid seed obtained from more traditional seed production practices.

Seed processing plant biological refuse can also be reduced using the methods described herein because there is less cob material with the development of the additional floret, as resources and tissue in the plant are being diverted from the mature cob structure to development of the additional floret.

Also provided are methods of increasing the efficiency of hybrid corn production by crossing a male and a female plant in which the female corn plant comprises one or more QTL alleles associated with the floral development variant phenotype at any of the QTL positions listed in Table 7, 8, 9, 10 or 11 and wherein the male corn plant does not have the QTL allele associated with the floret development variant phenotype.

Also provided is a method of creating the production female by introgressing a QTL allele associated with the floret development variant phenotype into a corn plant that does not comprise the QTL allele in its genome, said method comprising: A) providing at least a corn plant, B) genotyping at least one corn plant at one or more marker loci in any of the QTL regions listed in Table 7, 8, 9, 10, or 11, and, C) selecting at least one corn plant for further analysis or breeding based upon the presence of an allele at the at least one marker locus that is correlated with the QTL allele associated with the floret development variant phenotype.

All publications and published patent documents cited in this specification are incorporated herein by reference to the same extent as if each individual publication or patent application is specifically and individually indicated to be incorporated by reference.

### EXAMPLES

The examples described herein are meant to be representative and as examples of the discoveries, and are not limiting to the scope of the claims.

### Example 1

### Use of Floret Development Traits to Increase Seed Yields in F₁ Seed Production

A floret development variant donor (Inbred D) was generated from a public sweet corn line known as Country Gentleman (CG). Country Gentleman has a unique kernel development phenotype in that its kernels are arranged irregularly due to development of both the lower and upper florets, resulting in up to twice as many kernels per ear. Tassel development in this line is normal, and kernels are uniform, medium to small in size, and have a flat shape.

F₁ hybrid seed was produced by hand-crossing two elite inbred maize lines (Inbreds PHW6G and PH1CJB) with the floret development variant donor (Inbred D). The crosses were made bi-directionally, as diagrammed below in Table 1. The resulting F₁ hybrid genetics is identical regardless of the cross direction (i.e.: genetics of Inbred PHW6G/Inbred D and Inbred D/Inbred PHW6G are equal). Kernel orientation on the developing ear is determined by the genetics of the female (ear parent) plant.

TABLE 1
Diagram of bi-directional seed production.
A forward slash (/) indicates a manual controlled cross wherein
the parent before the slash is used as the female (ear parent)
and the parent after the slash is used as the male (pollen parent).

| Hybrid 1 | Hybrid 2 |
|---|---|
| Inbred PHW6G/Inbred D | Inbred PH1CJB/Inbred D |
| F1 Hybrid PHW6G/Inbred D | F1 Hybrid PH1CJB/Inbred D |
| Hybrid 3 | Hybrid 4 |
| Inbred D/Inbred PHW6G | Inbred D/Inbred PH1CJB |
| F1 Hybrid Inbred D/PHW6G | F1 Hybrid Inbred D/PH1CJB |

Seeds per pound data were collected for each of the bi-directional seed lots for both hybrids (Table 2). For Hybrid 1, the use of Inbred D as the female parent as compared to PHW6G resulted in 2484 more kernels per pound than Hybrid 3, amounting to an increase of 55%. For Hybrid 2, the use of Inbred D as the female parent as compared to PH1CJB resulted in 1506 more kernels per pound than Hybrid 4, amounting to an increase of 40%. Thus, seed yield was significantly increased when the floret development variant donor (Inbred D) was used as the female inbred parent in a hybrid combination.

TABLE 2
Kernels per pound data for two (2) bi-
directionally produced F₁ hybrids

| | Direction of cross | |
|---|---|---|
| | to produce F₁ | Kernels |
| Hybrid | hybrid seed | per pound |
| Hybrid 1 | PHW6G/Inbred D | 2062 |
| Hybrid 3 | Inbred D/PHW6G | 4545 |
| Hybrid 2 | PH1CJB/Inbred D | 2268 |
| Hybrid 4 | Inbred D/PH1CJB | 3774 |

Seeds of the F₁ Hybrids from each of the bi-directional crosses shown in Table 1 were planted and grown, and the resulting ears evaluated for kernel size and row orientation (Table 3). Regardless of whether or not the floret development variant donor was used as the female parent, kernel size was normal, as was the orientation of the rows of kernels on the ear.

TABLE 3
F₁ Hybrid ear observations for two (2)
bi-directionally produced F₁ hybrids.

| Hybrid | | Kernel | Row orientation |
|---|---|---|---|
| (seed | Direction of cross | Size (ear | of kernels (ear |
| source) | (seed source) | produced) | produced) |
| Hybrid 1 | PHW6G/Inbred D | Normal | Straight Rows |
| Hybrid 3 | Inbred D/PHW6G | Normal | Straight Rows |
| Hybrid 2 | PH1CJB/Inbred D | Normal | Straight Rows |
| Hybrid 4 | Inbred D/PH1CJB | Normal | Straight Rows |

### Example 2

### Effect of Floret Development Traits on Seed Production

Approximately 1000 ears from each of three populations (designated as Population A, Population B, and Population C) were scored as indicated in Table 4. In each population, Inbred D was crossed to an elite inbred. A score of “9” was given to ears that had normal kernel orientation in straight rows. A score of “7” was given to ears in which more than 50% of the kernels on the ear exhibited normal seed orientation; however, some of the kernels did exhibit loss of rowing. A score of “3” was given to ears in which more than 50% of the kernels on the ear were off-set and not in rows, however, some of the ear exhibited normal kernel row orientation. A score of “1” was given to ears that exhibited complete or nearly complete lack of kernel row orientation. Average kernels per pound were then determined for each score category for each of the three populations (Table 5). The observed increase in average kernels per pound due to the floret development trait was substantial in all three populations. The percent increase in kernels per pound comparing score “1” with score “9” within each population was as follows: 45% for Population A, 48% for Population B, and 22% for Population C.

TABLE 4
Description of kernel orientation scoring for corn
ear evaluation for floret development phenotype

| | Ear Score | | | |
|---|---|---|---|---|
| | 9 | 7 | 3 | 1 |
| Score | Kernel | >50% of the | <50% of the | Kernel |
| Description | orientation | kernel | kernel | orientation |
| | is complete | orientation | orientation | is lacking |
| | rows on | is in | is in | on nearly |
| | nearly the | complete | complete | the entire |
| | entire ear | rows | rows | ear |
| Biology of | No two- | Less than | More than | Two-floret |
| Description | floret | 50% of the | 50% of the | spikelet |
| | spikelet | ear exhibits | ear exhibits | development |
| | development | two-floret | two-floret | is apparent |
| | apparent | spikelet | spikelet | on nearly |
| | | development | development | 100% of the |
| | | | | ear |

TABLE 5
Seed size comparisons for maize floret development trait, as measured
in seeds per pound determination for three early generation populations,
ears grouped by kernel orientation scores.

| | Average | Average | Average | Average |
|---|---|---|---|---|
| | kernels | kernels | kernels | kernels |
| | per pound | per pound | per pound | per pound |
| | for Score | for Score | for Score | for Score |
| Population | of “9” | of “7” | of “3” | of “1” |
| Population A | 1839 | 2196 | 2431 | 2668 |
| Population B | 2002 | 2387 | 2569 | 2960 |
| Population C | 2032 | 2232 | 2196 | 2475 |
| Across A, B, | 1958 | 2272 | 2397 | 2701 |
| C | | | | |

### Example 3

### Use of Floret Development Traits to Increase Frequency of Small Seeds in Maize

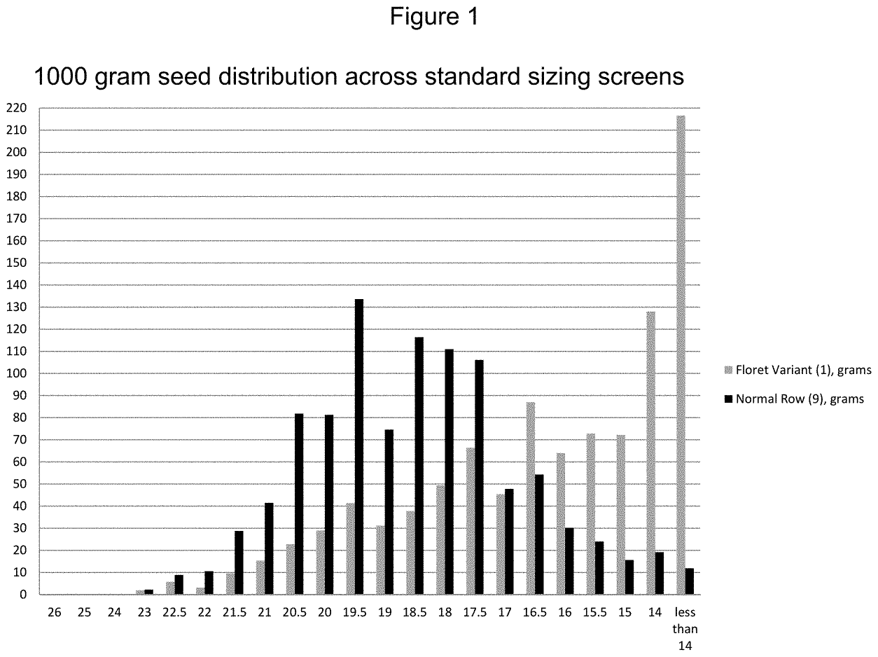

Ears from BC₀F₂ plants arising from a cross of Inbred 274 (normal floret development resulting in paired rows of kernels) with Inbred D (floret development variant donor) were harvested and then scored as shown in Table 4. Representative “1” ears were shelled and the kernels were bulked. Representative “9” ears were also shelled and the kernels were bulked. A 1000 gram sample of each bulk was then run through sizing screens. Sizing screens are routinely used in the seed industry to provide seedsmen with uniform metrics. The screens are calibrated and sized increments of 64^{th} of an inch from 26/64 to 23/64, followed by ½ 64^{th} increments. The smallest screen size is 14/64^{th} inch. The results of 1000 gram screen distribution for seed from floret development variant (Ear Score “1”) compared to the normal row (Ear Score “9”) are shown in Table 6. Data are also depicted visually in FIG. 1, which illustrates how the use of a floret development variant donor decreases seed size and increases uniformity. The floret development trait described in EXAMPLE 1 increases the frequency of small seeds.

TABLE 6
1000 Gram screen distribution for seed from floret
development variant (Ear Score “1”) compared
to the normal row (Ear Score “9”)

| Round Screen | Floret Variant | Normal Row |
|---|---|---|
| Size (64^{th} | (1), | (9), |
| of an inch | grams | grams |
| 26 | 0 | 0.2 |
| 25 | 0 | 0.2 |
| 24 | 0 | 0 |
| 23 | 2 | 2.3 |
| 22.5 | 5.8 | 8.9 |
| 22 | 3.2 | 10.6 |
| 21.5 | 9.6 | 28.8 |
| 21 | 15.4 | 41.5 |
| 20.5 | 22.8 | 81.8 |
| 20 | 29 | 81.3 |
| 19.5 | 41.4 | 133.6 |
| 19 | 31.2 | 74.6 |
| 18.5 | 37.8 | 116.4 |
| 18 | 49.4 | 111 |
| 17.5 | 66.4 | 106.1 |
| 17 | 45.4 | 47.8 |
| 16.5 | 87 | 54.3 |
| 16 | 64 | 30.2 |
| 15.5 | 72.8 | 24 |
| 15 | 72.2 | 15.7 |
| 14 | 128 | 19.2 |
| less than 14 | 216.6 | 11.9 |

### Example 4

### Mapping of Row Behavior QTLs Using Maize Biparental Populations

### Population Development for Initial Mapping

Two bi-parental populations were created, one from a cross between inbred D and B73 and the other from a cross between inbred D and Mo17, and the resulting F₁ progeny were selfed to create F₂ segregating populations. 745 F₂ plants from the Inbred D×B73 population and 803 F₂ plants from the inbred D×Mo17 population were genotyped with 328 and 318 SNP markers respectively. These markers were selected to detect recombinations occurring randomly across the 10 chromosomes. The markers were designed for use with TaqMan® SNP Genotyping Assay (Life Technology). The two populations were open pollinated and F₂ derived ears from each line were harvested and used for assessing scores for the rowing behavior trait. The degree of rowing was scored on a 1 to 9 scale, where ears with perfect rows, resembling the B73 or Mo17 parents, were given a score of 9. Ears showing more than half perfect rows were given a score of 7. Ears with about half of the kernels in a row and half distributed in a random fashion, due to the development of the two-kernel spikelets, were given a score of 5. Ears that expressed complete Country Gentleman rowing were given a score of 1. 616 and 707 ears for the Inbred D×B73 and Inbred D×Mo17 populations, respectively, were used for QTL analysis.

### QTL Analysis

QTL analyses were conducted using the composite interval mapping (CIM; Zeng, Z.-B. 1994. Precision mapping of quantitative trait loci. Genetics 136: 1457-146) function of the software suite Windows QTL Cartographer version 2.5_011 (http://statgen.ncsu.edu/qticart/WQTLCart.htm). For this analysis, the Haldane mapping function and a significance threshold LOD score of 3 was used. For the interval analysis, the maximum LOD value associated with the most closely linked marker, was tabulated (see last column in Tables 7 and 8). In the Inbred D×B73 population, 3 QTLs were identified as indicated in Table 7, while in the Inbred D×Mo17 population, 5 QTLs were identified as indicated in Table 8. For all of the QTLs, with the exception of the QTL on chromosome 2 in the Inbred D×Mo17 population, the allele contributing to the floral development variant phenotype comes from the Inbred D parent.

TABLE 7
QTL positions for Inbred D × B73 population

| QTL | Chromosome | Interval | Peak | LOD |
|---|---|---|---|---|
| 1 | 3 | 114-167 | 144.2 | 64.9 |
| 2 | 5 | 71-118 | 92.4 | 18.8 |
| 3 | 9 | 57-120 | 87.1 | 15.5 |

TABLE 8
QTL positions for Inbred D × Mo17 population

| QTL | Chromosome | Interval | Peak | LOD |
|---|---|---|---|---|
| 1 | 2 | 175-216 | 200.2 | 6.9 |
| 2 | 3 | 118-167 | 142.5 | 16.8 |
| 3 | 4 | 143-176 | 151.5 | 9 |
| 4 | 5 | 71-118 | 102.5 | 10.5 |
| 5 | 9 | 57-120 | 68.7 | 19 |

### Example 5

### Further Mapping of Row Behavior QTLs Using Maize Biparental Populations

### Population Development for Initial Mapping

Three F₂ populations were created for further validation by crossing inbred lines PHW6G, PH1D84, and PH1CJB to Inbred D. The resulting F₁ progeny were selfed to create three F₂ segregating populations. For each population, 368 F₂ plants were genotyped using 192 polymorphic markers. These markers were selected to detect recombinations occurring randomly across the 10 chromosomes. The markers were designed for use with TaqMan® SNP Genotyping Assay (Life Technology). The three populations were open pollinated and F₂ derived ears from each line were harvested and used for assessing scores for the rowing behavior trait. Ears that were diseased or runt were excluded from the analysis, leaving final mapping population sizes of 339, 343, and 334 individuals for populations PHW6G×Inbred D, PH1D84×Inbred D, and PH1CJB×Inbred D, respectively.

### QTL Analysis

QTL analyses were conducted using the composite interval mapping (CIM; Zeng, Z.-B. 1994. Precision mapping of quantitative trait loci. Genetics 136: 1457-146) function of the software suite Windows QTL Cartographer version 2.5_011 (http://statgen.ncsu.edu/qticart/WQTLCart.htm). For this analysis, the Haldane mapping function and a significance threshold LOD score of 3.0 was used. For the interval analysis, the maximum LOD value associated with the most closely linked marker, was tabulated (last column in Tables 9 and 10).

In the PHW6G×Inbred D population, four QTL were identified as indicated in Table 9. In the PH1D84×Inbred D population, five QTL were identified as indicated in Table 10. In the PH1CJB×Inbred D population, four QTL were identified as indicated in Table 11. For all of the QTL, the allele contributing to the lower row score was from inbred D.

Markers in these regions can be selected from publicly available sources, including but not limited to published papers, patents, and the Maize Genome Database.

TABLE 9
QTL positions for population PHW6G × Inbred D

| QTL | Chromosome | Interval | Peak | LOD |
|---|---|---|---|---|
| 1 | 1 | 87-123 | 104 | 4.1 |
| 2 | 3 | 120-168 | 148 | 34.2 |
| 3 | 5 | 87-118 | 106 | 4.3 |
| 4 | 9 | 43-107 | 69 | 3.7 |

TABLE 10
QTL positions for population PH1D84 × Inbred D

| QTL | Chromosome | Interval | Peak | LOD |
|---|---|---|---|---|
| 1 | 1 | 61-89 | 72 | 5.4 |
| 2 | 1 | 178-190 | 185 | 3.2 |
| 3 | 3 | 116-183 | 146 | 21.2 |
| 4 | 5 | 90-118 | 105 | 4.6 |
| 5 | 9 | 57-126 | 79 | 7.9 |

TABLE 11
QTL positions for population PH1CJB × Inbred D

| QTL | Chromosome | Interval | Peak | LOD |
|---|---|---|---|---|
| 1 | 3 | 56-91 | 75 | 5.6 |
| 2 | 3 | 126-153 | 140 | 21.5 |
| 3 | 4 | 89-117 | 116 | 3.9 |
| 4 | 5 | 118-148 | 130 | 5.3 |

### Example 6

### Introgression of Favorable QTL Alleles into Maize Plants

Introgressing a mapped locus into a plant which does not contain that locus requires several steps. In one method, markers can be found using publicly available databases such as the Maize Genome Database (available on the web) in order to find markers which may be different between the parental inbreds in the region or locus of interest. The parental inbreds are selected, one line which contains the locus of interest and one which you want to place the locus of interest within. These two lines are then crossed and possibly selfed or further backcrossed, in one of a myriad of possible breeding scenarios. The resulting offspring are then tested to determine which parental genomic locus is present in the regions of interest and then those plants comprising the region of interest are selected for further breeding. In other methods, one could use double haploid strategies to decrease generation time and create inbreds comprising the introgressed region of interest, while at the same time comprising favorable genetic compositions from the other parent in the original cross.

### Example 7

### Parent Offspring Trait and Marker Validation

F₃ seed from the Inbred D×B73 population was planted in a winter nursery for the purpose of seed increase. F₃ seed was selected for planting if characteristics of the floret development variant phenotype were exhibited. F₃ plants were grown to maturity and ears were self-pollinated manually. Genotypic data was also obtained. The experiment showed that selections in the previous generation greatly enriched the resulting population to have more #1s on the rowing scale (see Example 2). Moreover, the same QTL identified in Table 7 were observed.

What is claimed is:

1. Field corn ears containing kernels with non-uniform rowing and uniform seeds of small to medium size and flat shape.

2. Seed of the field corn ears of claim 1.

3. The seed of claim 2, further comprising a seed treatment.

4. A method of hybrid seed production for field corn, said method comprising:
a. planting floret development variant donors as females in a hybrid corn production field;
b. harvesting ears from the females; and
c. obtaining hybrid field corn seed from the ears.

5. The method of claim 4, wherein said floret development variant donors are inbreds or modified inbreds.

6. The method of claim 4, wherein said floret development variant donors are detasseled prior to anthesis.

7. The method of claim 4, wherein said floret development variant donors are male-sterile.

8. The method of claim 4, wherein said method produces uniform seed lots of small to medium flat seeds.

9. The method of claim 4, wherein said method increases seed production yield.

10. The method of claim 4, wherein said method minimizes post-harvest processing damage to seed.

11. The method of claim 4, wherein said method reduces seed drying time.

12. The method of claim 4, wherein said method facilitates the removal of the seed from the ears.

13. The method of claim 4, wherein said method decreases seed processing equipment wear and tear and maintenance.

14. The method of claim 4, wherein said method reduces or eliminates the need for seed size sorting.

15. The method of claim 4, wherein said method increases seed treatment uniformity and efficiency.
